(19) United States
(12) Patent Application Publication
(10) Pub. No.: US 2009/0311354 A1
(43) Pub. Date: Dec. 17, 2009

(54) COMPOSITION COMPRISING EXTRACTS OR FRACTIONS OF MAGNOLIA OBOVATA THUNB FOR TREATING AND PREVENTING INFLAMMATION DISEASE

(75) Inventors: Byoung-Mog KWON, DAEJEON (KR); Dong Cho HAN, DAEJEON (KR); Hye-Nan KIM, DAEJEON (KR); Young-Min HAN, DAEJEON (KR); So Young LEE, DAEJEON (KR); Dae-Seop SHIN, Koysan-gun, Chungcheongbuk-do (KR)

Correspondence Address: LUCAS & MERCANTI, LLP, 475 PARK AVENUE SOUTH, 15TH FLOOR, NEW YORK, NY, 10016, US

(73) Assignee: KOREA RESEARCH INSTITUTE OF BIOSCIENCE AND BIOTECHNOLOGY, Daejeon (KR)

(21) Appl. No.: 12/198,553

(22) Filed: Aug. 26, 2008

(30) Foreign Application Priority Data
Jun. 11, 2008 (KR) 10-2008-0054621

(51) Int. Cl. A61K 36/575; A61K 31/09; A61P 29/00

(52) U.S. Cl. 424/777; 424/725; 424/778; 514/718

(57) ABSTRACT

The present invention relates to a composition for the prevention and treatment of inflammatory disease containing the extracts of Magnolia obovata or fractions thereof as an active ingredient, more precisely, a composition for the prevention and treatment of inflammatory disease containing the extracts of Magnolia obovata fruits and floral buds extracted with alcohol or alcohol aqueous solution as a solvent and active fractions isolated from the same. The extracts and fractions of the present invention inhibit lipopolysaccharide (LPS) induced nitric oxide (NO) generation significantly, have anti-inflammation activity and low cytotoxicity, and contain all of major effective components isolated from Magnolia obovata such as obovatol, honokiol and magnolol, so that they can be effectively used for the prevention and treatment of inflammatory disease.

### TECHNICAL FIELD

The present invention relates to a composition containing the extract of Magnolia obovata or fractions thereof as an active ingredient for the prevention and treatment of inflammatory disease, more precisely a composition containing the extract of Magnolia obovata extracted from fruits or floral buds of the same with alcohol or alcohol aqueous solution as a solvent or active fractions isolated from the extract as an active ingredient for the prevention and treatment of inflammatory disease.

### BACKGROUND ART

Inflammatory reaction is induced when tissues (cells) are damaged or infected by the external source of infection (bacteria, fungi, virus or various allergens). At this time, a series of complicated physiological reactions mediated by various inflammatory mediators in local blood vessels and body fluids and immune cells are induced such as enzyme activation, secretion of inflammatory mediators, body fluid infiltration, cell migration and tissue destruction, etc, along with symptoms such as erythema, edema, pyrexia and pain. Normally, inflammatory reaction is to eliminate the external source of infection and regenerate the damaged tissue to recover the functions in life. However, if an antigen is not eliminated or an endogenous material is the cause of inflammation, so that inflammatory reaction is excessive or continues long, mucous membrane damage is accelerated and as a result, in some cases, it causes another disease including cancer (Jonathan Cohen, Nature, 420, 885-891, 2002).

Various in vivo biochemical phenomena are involved in the development of inflammation. In particular, nitric oxide synthase (NOS) which is the enzyme generating nitric oxide (NO) and enzymes involved in biosynthesis of prostaglandin are important mediators for inflammatory reaction. Therefore, NOS, the enzyme generating NO from L-arginine, or cyclooxygenase (COX), the enzyme involved in the synthesis of prostaglandin from arachidonic acid are major targets for inhibition of inflammation.

According to the recent studies, there are different types of NOS, for example brain NOS (bNOS) found in the brain, neuronal NOS (nNOS) found in the nervous system, and endothelial NOS (eNOS) found in the blood vessel system. These are all regularly expressed in vivo and a small amount of NO endogenously generated by the enzymes plays an important role in maintaining homeostasis by inducing neurotransmission or vasodilation. On the contrary, excessive NO generated by iNOS (induced NOS) induced by various cytokines or a foreign stimulus causes cytotoxicity and various inflammatory reactions. In particular, chronic inflammation is closely related to the increase of iNOS activity (Jon O. Lundberg, et al., Nature Reviews Drug Discovery, 2008). COX has two different isotypes. COX-1 exists always in cells to play a certain role in synthesis of prostaglandin (PGs) necessary for the protection of cells, while COX-2 increases rapidly in cells by an inflammatory stimulus and then plays an important role in inducing inflammatory reaction. Transcription inflammatory factors including iNOS and COX-2 which increase the levels of NO and PGs are one of the causes of chronic diseases including sclerosis, Parkinson's disease, Alzheimer's disease and colon cancer (Bengt Samuelsson, et al., Pharmacological Reviews, 59, 207-224, 2007).

Lipopolysaccharide (LPS) is an extracellular secreted bacteria toxin, which stimulates inflammatory reaction and mediates secretions of various inflammatory regulators such as NO, cytokine, TNF—, prostaglandin E2 and eicosanoid accelerating inflammatory reaction (Chen YC, et al., Biochem. Pharmacol., 61, 1417-1427, 2001). NO synthase is divided into two groups; cNOS, for an example, exists in cells (for example, neurons and endothelial cells) at a regular level and its transcription is regulated by calcium dependent calmodulin. In the meantime, NOS in smooth muscle cells, macrophages, hepatocytes and astrocytes is induced by inflammatory cytokines and lipopolysaccharide. The activation of NOS is a critical factor in the development of various inflammatory diseases since it accelerates generation of a large amount of NO. Thus, the generation of NO induced by NOS can be an index to determine the degree of inflammation and the progress of inflammation. Expressions of specific genes such as COX-2 and iNOS, interleukin-1, interleukin-2, interleukin-6 and TNF are most concerned (Csaba Szabo, et al., Nature Reviews Drug Discovery, 6, 662-680. 2007).

Magnolia obovata has been used as a medicinal herb in folk remedy, which contains 1-2% of essential oils. Major components of the tree are lignan compounds such as honokiol, magnolol and obovatol, which have been known to have anti-bacterial and anti-cancer activities (H. Matstsuda, et al., Chem. Pharm. Bull. 49, 716-720, 2001; Kwon, B. M. et al, Korean Patent No. 697236; Kwon, B. M. et al, Planta Medica, 63, 550-551, 1997; Hwang, E. I, et al., Antimicrob. Chemotherapy 49, 95-101, 2002; Myoung Suk Choi, et at., European Journal of Pharmacology 556, 181.189, 2007). However, there has been no report on the anti-inflammation effect of fruit or floral bud extract of Magnolia obovata Thunberg (Magnoliaceae) or fractions thereof

Thus, the present inventors studied and disclosed that the fruit or floral bud extract of Magnolia obovata Thunberg (Magnoliaceae) or fractions isolated therefrom have anti-inflammation effect by inhibiting lipopolysaccharide (LPS) mediated nitric oxide (NO) generation, leading to the completion of this invention.

### DISCLOSURE

### Technical Problem

It is an object of the present invention to provide a composition containing the extract of Magnolia obovata fruits or floral buds and active fractions isolated therefrom as an active ingredient for the prevention and treatment of inflammatory disease, and to provide a health improving functional food for the prevention and improvement of inflammatory disease.

### Technical Solution

To achieve the above object, the present invention provides a composition containing the extracts of Magnolia obovata as an active ingredient for the prevention and treatment of inflammatory disease.

The present invention also provides a composition containing active fractions isolated from the extracts of Magnolia obovata as an active ingredient for the prevention and treatment of inflammatory disease.

The present invention further provides a method for the treatment of inflammatory disease containing the step of administering the extracts of Magnolia obovata or active fractions isolated therefrom to a subject with inflammatory disease.

The present invention further provides a method for the prevention of inflammatory disease containing the step of administering the extracts of Magnolia obovata or active fractions isolated therefrom to a subject with inflammatory disease.

The present invention also provides a use of the extracts of Magnolia obovata or active fractions thereof for the preparation of a composition for the prevention and treatment of inflammatory disease.

The present invention also provides a health improving functional food containing the extracts of Magnolia obovata or active fractions thereof for the prevention and improvement of inflammatory disease.

In addition, the present invention provides a use of the extracts of Magnolia obovata or active fractions thereof for the preparation of a health improving functional food for the prevention and improvement of inflammatory disease.

Hereinafter, terms used in this invention are described.

“Prevention” herein indicates every action to delay the development of inflammatory disease by administering the composition of the present invention.

“Treatment” or “improvement” indicates every action to improve or induce advantageous changes in the said disease by administering the composition of the present invention.

“Administration” herein indicates providing a pharmaceutically effective dose of the composition of the present invention to a subject according to a proper method.

“Subject” herein indicates a human or an animal such as ape, dog, goat, pig or rat that can be improved from the said disease by the administration of the composition of the present invention.

“Pharmaceutically effective dose” herein indicates the amount of the composition of the present invention which is enough to treat disease and formulated according to reasonable receiving ratio or risk ratio for clinical application. This amount can be determined considering various factors such as kind of a disease, severity of a disease, activity of a drug, sensitivity to a drug, administration time and pathway, elimination rate, term of treatment, drugs co-used, and other factors well-known to those in medical field.

Hereinafter, the present invention is described in detail.

The present invention provides a composition containing the extracts of Magnolia obovata or active fractions thereof as an active ingredient for the prevention and treatment of inflammatory disease.

The Magnolia obovata above is Magnolia obovata Thunberg (Magnoliaceae), but not always limited thereto.

The extract of Magnolia obovata and active fractions thereof are preferably isolated from fruits or floral buds of the tree, but not always limited thereto.

The extract of Magnolia obovata and active fractions thereof preferably contain all of obovatol represented by formula 1, honokiol represented by formula 2 and magnolol represented by formula 3, but not always limited thereto.

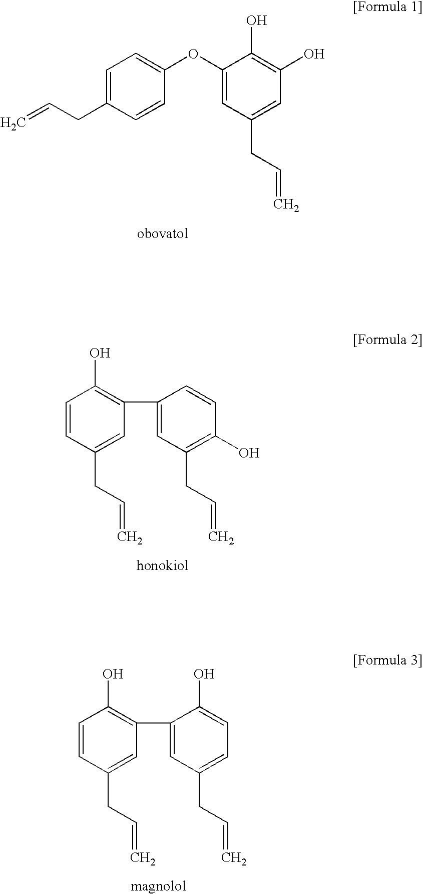

The inflammatory disease is preferably selected from the group consisting of gastritis, colitis, arthritis, nephritis, hepatitis and degenerative disease, but not always limited thereto.

The extracts of Magnolia obovata or active fractions thereof of the present invention are preferably prepared by the method comprising the following steps, but not always limited thereto:

1) soaking dried fruits or floral buds of Magnolia obovata in C₁-C₄ lower alcohol or alcohol aqueous solution and then extracting the Magnolia obovata extract; and

2) concentrating the Magnolia obovata extract prepared in step 1) under reduced pressure, to which an organic solvent is added, followed by column chromatography to give active fractions.

In this method, the Magnolia obovata of step 1) is preferably Magnolia obovata Thunberg (Magnoliaceae) and can be either cultivated or purchased.

In this method, the lower alcohol of step 1) is preferably ethanol and more preferably methanol, but not always limited thereto. Water, alcohol or the mixture thereof was added to the dried fruits or floral buds of Magnolia obovata by 2-5 times the weight of the fruits or floral buds, followed by extraction, and more preferably the solvent is added by 2-3 times the weight of the fruits or floral buds, but not always limited thereto. The temperature for the extraction is preferably 30-100° C., and more preferably 50-80° C., but not always limited thereto. The extraction time is preferably 1-5 days, and more preferably 2-3 days, but not always limited thereto. After extracting by the above method, the extract is filtered and concentrated under reduced pressure to give the extract of Magnolia obovata, but not always limited thereto.

In this method, the column chromatography of step 2) can be performed by using the column filled with a filler selected from the group consisting of silica gel, sephadex, RP-18, polyamide, Toyopearl and XAD resin for the isolation and purification. The column chromatography using a proper filler can be repeated several times. And ethyl acetate-hexane can be used as a solvent, but not always limited thereto.

The present inventors soaked the dried fruits and floral buds of Magnolia obovata (Magnoliaceae) in ethanol, methanol and methanol aqueous solution respectively, which stood at room temperature for 48 hours. Then, the mixture was filtered and concentrated under reduced pressure to give the extracts of Magnolia obovata. After dissolving the extracts in different organic solvents, silica gel is added thereto to absorb active materials. Fractions obtained from silica gel column chromatography (ethyl acetate:hexane=10:90-20:80) were analyzed by thin layer chromatography (eluent, ethyl acetate:hexane=3:7).

HPLC was performed to analyze the extracts of Magnolia obovata (Magnoliaceae) fruits and floral buds and active fractions isolated from the same. As a result, the extracts of Magnolia obovata (Magnoliaceae) fruits and floral buds and active fractions thereof contained all of obovatol, honokiol and magnolol. The extracts of Magnolia obovata Thunberg reported previously contained obovatol as a major component and honokiol and magnolol were included very small amounts. In the mean time, the extracts of the present invention prepared from fruits and floral buds of Magnolia obovata Thunberg (Magnoliaceae) and fractions thereof have been confirmed to contain all of obovatol, honokiol and magnolol by large amounts.

To investigate cytotoxicity of the extracts of Magnolia obovata Thunberg (Magnoliaceae) fruits and floral buds and active fractions thereof, MTT assay (3-[4,5-dimethylthiazlyl]-2,5-diphenyl tetrazolium bromide) was performed. To do so, RAW264.7 cells were treated with the extracts of Magnolia obovata Thunberg (Magnoliaceae) fruits and floral buds and active fractions thereof at different concentrations and then cultured, followed by MTT assay. As a result, IC₅₀ values of the extracts of Magnolia obovata Thunberg (Magnoliaceae) fruits and floral buds and active fractions thereof of the present invention were all up to 50 μg/ml, suggesting that they have very low cytotoxicity (see Table 2).

The present inventors also investigated whether or not the extracts of Magnolia obovata fruits and floral buds and active fractions thereof could inhibit NO generation which is closely related to inflammatory reaction. RAW264.7 cells were cultured in media treated with different concentrations of the extracts of Magnolia obovata fruits and floral buds and active fractions thereof with or without lipopolysaccharide, and then the accumulation of nitrates in the culture solution was measured by Griess test. As a result, the extracts of Magnolia obovata fruits and floral buds and active fractions thereof significantly reduced lipopolysaccharide induced NO generation dose-dependently.

Therefore, the extracts of Magnolia obovata fruits and floral buds and active fractions thereof of the present invention can be effectively used as a composition for the prevention and treatment of inflammatory disease by inhibiting lipopolysaccharide induced NO generation in inflammatory cells without cytotoxicity.

The composition for the prevention and treatment of inflammatory disease of the present invention can contain the extracts of Magnolia obovata, active fractions isolated therefrom or the mixture thereof and additionally one or more active ingredients having the same or similar functions to the above.

The composition of the present invention contains the extracts of Magnolia obovata, active fractions isolated therefrom or the mixture thereof by 0.1-50 weight % by the total weight of the composition, but not always limited thereto.

The extracts of Magnolia obovata, active fractions isolated therefrom or the mixture thereof of the present invention can be administered orally or parenterally and be used in general forms of pharmaceutical formulation. The composition of the present invention can be prepared for oral or parenteral administration by mixing with generally used diluents or excipients such as fillers, extenders, binders, wetting agents, disintegrating agents and surfactant. Solid formulations for oral administration are tablets, pills, powders, granules and capsules. These solid formulations are prepared by mixing the pharmaceutical composition of the present invention with one or more suitable excipients such as starch, calcium carbonate, sucrose or lactose, gelatin, etc. Liquid formulations for oral administrations are suspensions, solutions, emulsions and syrups, and the above-mentioned formulations can contain various excipients such as wetting agents, sweeteners, aromatics and preservatives in addition to generally used simple diluents such as water and liquid paraffin. Formulations for parenteral administration are sterilized aqueous solutions, water-insoluble excipients, suspensions, emulsions, lyophilized preparations, suppositories and injections. Water insoluble excipients and suspensions can contain, in addition to the active compound or compounds, propylene glycol, polyethylene glycol, vegetable oil like olive oil, injectable ester like ethylolate, etc. Suppositories can contain, in addition to the active compound or compounds, witepsol, macrogol, tween 61, cacao butter, laurin butter, glycerol, gelatin, etc. The composition of the present invention can be administered by parenterally and the parenteral administration includes subcutaneous injection, intravenous injection and intramuscular injection.

The dosage units can contain, for example, 1, 2, 3 or 4 individual doses or ½, ⅓ or ¼ of an individual dose. An individual dose preferably contains the amount of active compound which is administered in one application and which usually corresponds to a whole, ½, ⅓ or ¼ of a daily dose. The effective dosage of the composition of the present invention is 5 mg˜100 mg/kg per day and preferably 5 mg˜50 mg/kg per day, and administration frequency is 1˜6 times a day. However the effective dosage can be changed according to administration pathway, severity of a disease, gender, weight and age. Therefore, the dosage cannot limit the scope of the present invention by any means.

The present invention also provides a method for the treatment of inflammatory disease containing the step of administering the pharmaceutically effective dosage of the extract of Magnolia obovata or active fractions thereof to a subject with inflammatory disease.

The present invention also provides a method for the prevention of inflammatory disease containing the step of administering the pharmaceutically effective dosage of the extract of Magnolia obovata or active fractions thereof to a subject with inflammatory disease.

The present invention also provides a use of the extracts of Magnolia obovata or active fractions thereof for the preparation of a composition for the prevention and treatment of inflammatory disease.

The inflammatory disease is preferably selected from the group consisting of gastritis, colitis, arthritis, nephritis, hepatitis and degenerative disease, but not always limited thereto.

The present invention also provides a health improving functional food containing the extracts of Magnolia obovata or active fractions thereof for the prevention and improvement of inflammatory disease.

In addition, the present invention provides a use of the extracts of Magnolia obovata or active fractions thereof for the preparation of a health improving functional food for the prevention and improvement of inflammatory disease.

The Magnolia obovata above is Magnolia obovata Thunberg (Magnoliaceae), but not always limited thereto.

The extracts of Magnolia obovata and active fractions thereof are preferably isolated from the Magnolia obovata fruits or floral buds, but not always limited thereto.

The extracts of Magnolia obovata and active fractions thereof preferably contain all of obovatol represented by formula 1, honokiol represented by formula 2 and magnolol represented by formula 3, but not always limited thereto.

The inflammatory disease is preferably selected from the group consisting of gastritis, colitis, arthritis, nephritis, hepatitis and degenerative disease, but not always limited thereto.

The extracts of Magnolia obovata, active fractions isolated therefrom or the mixture thereof of the present invention can be used as a food additive. In that case, the extracts of Magnolia obovata, active fractions isolated therefrom or the mixture thereof can be added as they are or as mixed with other food components according to the conventional method. It is preferred to extract Magnolia obovata by using hot water or ethanol and at this time the preferable concentration of ethanol is 50-70%. The mixing ratio of active ingredients can be regulated according to the purpose of use (prevention, health enhancement or treatment). In general, to produce health food or beverages, the extracts of Magnolia obovata, active fractions isolated therefrom or the mixture thereof of the present invention are added preferably by up to 15 weight part and more preferably by up to 10 weight part. However, if long term administration is required for health and hygiene or regulating health condition, the content can be lower than the above but higher content can be accepted as well since the extracts of Magnolia obovata, active fractions isolated therefrom or the mixture thereof of the present invention have been proved to be very safe.

The food herein is not limited. For example, the extracts of Magnolia obovata, active fractions isolated therefrom or the mixture thereof of the present invention can be added to meats, sausages, breads, chocolates, candies, snacks, cookies, pizza, ramyuns, flour products, gums, dairy products including ice cream, soups, beverages, tea, drinks, alcohol drinks and vitamin complex, etc, and in wide sense, almost every food applicable in the production of health food can be included.

The composition for health beverages of the present invention can additionally include various flavors or natural carbohydrates, etc, like other beverages. The natural carbohydrates above can be one of monosaccharides such as glucose and fructose, disaccharides such as maltose and sucrose, polysaccharides such as dextrin and cyclodextrin, and glucose alcohols such as xilytole, sorbitol and erythritol. Besides, natural sweetening agents such as thaumatin and stevia extract, and synthetic sweetening agents such as saccharin and aspartame can be included as a sweetening agent. The preferable content of the natural carbohydrates in the composition of the present invention is 0.01-0.04 g per 100 ml and 0.02-0.03 g per 100 ml is more preferred.

In addition to the ingredients mentioned above, the extracts of Magnolia obovata, active fractions isolated therefrom or the mixture thereof of the present invention can include in variety of nutrients, vitamins, minerals, flavors, coloring agents, pectic acid and its salts, alginic acid and its salts, organic acid, protective colloidal viscosifiers, pH regulators, stabilizers, antiseptics, glycerin, alcohols, carbonators which used to be added to soda, etc. The extracts of Magnolia obovata, active fractions isolated therefrom or the mixture thereof of the present invention can also include natural fruit juice, fruit beverages and/or fruit flesh addable to vegetable beverages. All the mentioned ingredients can be added singly or together.

### Advantageous Effect

The extracts of Magnolia obovata fruits and floral buds or active fractions thereof of the present invention significantly inhibit lipopolysaccharide mediated NO generation, suggesting that they have anti-inflammatory activity, and have low cytotoxicity but high contents of obovatol, honokiol and magnolol, so that they can be effectively used as a composition for the prevention and treatment of inflammatory disease or as a functional health food.

### MODE FOR INVENTION

Practical and presently preferred embodiments of the present invention are illustrative as shown in the following Examples.

However, it will be appreciated that those skilled in the art, on consideration of this disclosure, may make modifications and improvements within the spirit and scope of the present invention.

### EXAMPLE 1

### Preparation of Extracts of Magnolia obovata

<1-1> Preparation of Methanol Extract of Magnolia obovata Fruits

4 L of methanol (22-25° C.) was added to 1 kg of the dried Magnolia obovata Thunberg (Magnoliaceae) fruits (picked up directly in Daejeon, Korea: September), which stood at room temperature for 48 hours. After stirring, the mixture was filtered with a filter paper and liquid phase and solid part were separated. The liquid phase was concentrated under reduced pressure, and the concentrate was dissolved in methanol. The organic solvent layer containing active materials was concentrated under reduced pressure. As a result, 40.5 g of methanol extract of Magnolia obovata Thunberg fruits was obtained.

<1-2> Preparation of Methanol Extract of Magnolia obovata Floral Buds

Extraction was performed by the same manner as described in Example <1-1> except that floral buds (picked up directly in Daejeon, Korea: December) of Magnolia obovata Thunberg (Magnoliaceae) were used instead of fruits of the tree. As a result, 40.2 g of methanol extract of Magnolia obovata Thunberg floral buds was obtained.

<1-3> Preparation of Ethanol Extract of Magnolia obovata Fruits

Extraction was performed by the same manner as described in Example <1-1> except that ethanol was used for the extraction instead of methanol. As a result, 39.5 g of ethanol extract of Magnolia obovata Thunberg fruits was obtained.

<1-4> Preparation of Ethanol Extract of Magnolia obovata Floral Buds

Extraction was performed by the same manner as described in Example <1-2> except that ethanol was used for the extraction instead of methanol. As a result, 38.8 g of ethanol extract of Magnolia obovata Thunberg floral buds was obtained.

<1-5> Preparation of 70% Methanol Aqueous Solution Extract of Magnolia obovata Fruits

Extraction was performed by the same manner as described in Example <1-1> except that 70% methanol aqueous solution was used for the extraction instead of methanol. As a result, 39.0 g of methanol aqueous solution extract of Magnolia obovata Thunberg fruits was obtained.

### EXAMPLE 2

### Preparation of Active Fractions of the Extracts of Magnolia obovata

<2-1> Preparation of Fractions of Methanol Extract of Magnolia obovata Fruits

10 g of the methanol extract of Magnolia obovata Thunberg (Magnoliaceae) fruits prepared in Example <1-1> was dissolved in methylene chloride, to which 10 g of silica gel (Merck, Art No. 9385) was added to absorb active materials. The ratio of ethyl acetate to hexane was changed from 10:90 to 20:80. Fractions obtained from silica gel column chromatography were analyzed by thin layer chromatography (eluent, ethyl acetate:hexane=3:7). As a result, 2.2 g of active fractions was obtained.

<2-2> Preparation of Fractions of Methanol Extract of Magnolia obovata Floral Buds

Extraction was performed by the same manner as described in Example <2-1> except that 10 g of the methanol extract of Magnolia obovata Thunberg (Magnoliaceae) floral buds was used instead of the methanol extract of Magnolia obovata Thunberg (Magnoliaceae) fruits. As a result, 1.8 g of fractions was obtained.

<2-3> Preparation of Fractions of Ethanol Extract of Magnolia obovata Fruits

Extraction was performed by the same manner as described in Example <2-1> except that 10 g of the ethanol extract of Magnolia obovata Thunberg (Magnoliaceae) fruits was used instead of the methanol extract of Magnolia obovata Thunberg (Magnoliaceae) fruits. As a result, 2.1 g of fractions was obtained.

<2-4> Preparation of Fractions of Ethanol Extract of Magnolia obovata Floral Buds

Extraction was performed by the same manner as described in Example <2-1> except that 10 g of the ethanol extract of Magnolia obovata Thunberg (Magnoliaceae) floral buds was used instead of the methanol extract of Magnolia obovata Thunberg (Magnoliaceae) fruits. As a result, 1.5 g of fractions was obtained.

### EXAMPLE 3

### Analysis of Components of the Extracts of Magnolia obovata and Fractions Thereof

The extracts of Magnolia obovata Thunberg (Magnoliaceae) fruits and floral buds and fractions thereof obtained in Example 1 and Example 2 proceeded to HPLC for analyzing their components. Conditions for HPLC were as shown in Table 1 (Table 1).

TABLE 1

| | |
|---|---|
| Factor | Condition |
| Manufacturer | Hewlett Packard, USA |
| Column | YMC, J□sphere ODS-H80, 250 × 20 mm I.D, |
| | S-4 uM, 8 nm |
| Moving phase | 80% MeOH: 20% water |
| Moving rate | 4 ml/min |

As a result, it was confirmed that the extracts of Magnolia obovata Thunberg (Magnoliaceae) fruits and floral buds and fractions thereof contained all of obovatol, honokiol and magnolol.

### EXPERIMENTAL EXAMPLE 1

### Cytotoxicity Test

RAW264.7 cells, the mouse macrophage-like cells, were purchased from American type culture collection (Cryosite, Lane Cove NSW, Australia). DMEM, penicillin, streptomycin and FBS were purchased from Gibco Life Technology (Rockville, Md., USA). The RAW264.7 cells were cultured in DMEM supplemented with 10% FBS, 100 U/ml penicillin and 100 μg/ml streptomycin in a 37° C. 5% CO₂ incubator. The cultured cells were inoculated in a 96-well plate at the concentration of 10⁴ cells/well, followed by further culture. Cytotoxicity was investigated by MTT assay (3-[4,5-dimethylthiazlyl]-2,5-diphenyl tetrazolium bromide) at the 24^{th} hour, 48^{th} hour and 72^{nd} hour of culture. To quantify the metabolic activity, OD₅₇₀ was measured.

As a result, the extracts and fractions of the present invention were all confirmed to have very low cytotoxicity, as shown in Table 2.

TABLE 2

| Experimental group | Cytotoxicity (IC₅₀ μg/ml) |
|---|---|
| Extract of Example <1-1> | <50 |
| Extract of Example <1-2> | <50 |
| Extract of Example <1-3> | <50 |
| Extract of Example <1-4> | <50 |
| Extract of Example <1-5> | <50 |
| Fraction of Example <2-1> | <50 |
| Fraction of Example <2-2> | <50 |
| Fraction of Example <2-3> | <50 |
| Fraction of Example <2-4> | <50 |

### EXPERIMENTAL EXAMPLE 2

### Inhibition of Lipopolysaccharide Induced NO Generation in RAW264.7 Cells

To investigate inhibitory activity of the extracts of Magnolia obovata Thunberg (Magnoliaceae) fruits and floral buds or active fractions thereof on lipopolysaccharide (LPS) induced nitric oxide (NO) generation in RAW264.7 cells, nitrate accumulation in the cell culture solution was examined by Griess reaction. Particularly, RAW264.7 cells were cultured in a 96-well plate at the concentration of 2×10⁵ cells/well. The extracts of Magnolia obovata Thunberg (Magnoliaceae) fruits and floral buds and fractions thereof were treated to media with or without lipopolysaccharide (1 μg/ml) at different concentrations of 1, 5, 10, 25, and 50 μg/ml, followed by culture for 24 hours. Accumulation of nitrates was measured by Griess reaction test. Equal amount of Griess reagent [0.1% N-(1-naphthyl)-ethylenediamine, 1% sulfanilamide in 5% phosphoric acid] was added to 50 μl of each culture solution, which stood at room temperature for 10 minutes. OD₅₅₀ was measured and nitrate in the culture solution was quantified by a standard curve made by OD of the known concentration of nitrates.

As a result, when lipopolysaccharide was co-treated with the extracts of Magnolia obovata Thunberg (Magnoliaceae) fruits and floral buds to the cells for 24 hours, lipopolysaccharide induced nitrate accumulation was significantly reduced in the culture solution the extract and/or the fraction dose-dependently. IC₅₀ of lipopolysaccharide induced NO generation was 8˜10 μg/ml. In Example 3, the extracts of Magnolia obovata Thunberg (Magnoliaceae) and fractions thereof did not exhibit cytotoxicity against inflammatory cells at the concentration of 50 μg/ml, suggesting that the extracts and fractions could inhibit lipopolysaccharide induced NO generation without cytotoxicity.

The Manufacturing Examples of the composition for the present invention are described hereinafter.

### MANUFACTURING EXAMPLE 1

### Preparation of Pharmaceutical Formulations

<1-1> Preparation of powders

| | |
|---|---|
| Extract of Example <1-1> | 2 g |
| Lactose | 1 g |

Powders were prepared by mixing all the above components, which were filled in airtight packs according to the conventional method for preparing powders.

<1-2> preparation of tablets

| | |
|---|---|
| Extract of Example <1-1> | 100 mg |
| Corn starch | 100 mg |
| Lactose | 100 mg |
| Magnesium stearate | 2 mg |

Tablets were prepared by mixing all the above components by the conventional method for preparing tablets.

<1-3> Preparation of capsules

| | |
|---|---|
| Extract of Example <2-1> | 100 mg |
| Corn starch | 100 mg |
| Lactose | 100 mg |
| Magnesium stearate | 2 mg |

Capsules were prepared by mixing all the above components, which were filled in gelatin capsules according to the conventional method for preparing capsules.

<1-4> Preparation of pills

| | |
|---|---|
| Extract of Example <1-2> | 1 g |
| Lactose | 1.5 g |
| Glycerin | 1 g |
| Xylitol | 0.5 g |

Pills were prepared by mixing all the above components according to the conventional method for preparing pills. Each pill contained 4 g of the mixture.

<1-5> Preparation of granules

| | |
|---|---|
| Extract of Example <2-2> | 150 mg |
| Soybean extract | 50 mg |
| Glucose | 200 mg |
| Starch | 600 mg |

All the above components were mixed, to which 100 mg of 30% ethanol was added. The mixture was dried at 60° C. and the prepared granules were filled in packs.

### MANUFACTURING EXAMPLE 2

### Preparation of Foods

Foods containing the extracts or fractions of the present invention were prepared as follows.

### <2-1> Preparation of Spices for Cooking

Health enhancing spices for cooking was prepared with 20˜95 weight part of the extract of Example <1-3> according to the conventional method.

### <2-2> Preparation of Tomato Ketchup and Sauce

Health enhancing tomato ketchup or sauce was prepared by mixing 0.2˜1.0 weight part of the extract of Example. <1-3> with tomato ketchup or sauce according to the conventional method.

### <2-3> Preparation of Flour Food

0.5˜5.0 weight part of the extract of Example <1-3> was added to the flour. Health enhancing foods such as bread, cake, cookies, crackers and noodles were prepared with the flour mixture according to the conventional method.

### <2-4> Preparation of Soups and Gravies

0.1˜5.0 weight part of the extract of Example <1-4> was added to soups and gravies. Health enhancing meat products, soups and gravies were prepared with this mixture by the conventional method.

### <2-5> Preparation of Ground Beef

Health enhancing ground beef was prepared by mixing 10 weight part of the extract of Example <1-4> with ground beef according to the conventional method.

### <2-6> Preparation of Dairy Products

5˜10 weight part of the fraction of Example <2-3> was added to milk. Health enhancing dairy products such as butter and ice cream were prepared with the milk mixture according to the conventional method.

### <2-7> Preparation of Sun-Sik

Brown rice, barley, glutinous rice and Yulmu (Job's tears) were gelatinized according to the conventional method, dried and pulverized to obtain 60-mesh powders.

Black soybean, black sesame and wild sesame were steamed and dried according to the conventional method and pulverized to obtain 60-mesh powders.

The fraction of Example <2-3> was concentrated under reduced pressure, spray-dried and pulverized to obtain 60-mesh dry powders.

Sun-Sik was prepared by mixing the dry powders of the grains, seeds and the fraction of Example <2-3> according to the below ratio.

Grains (brown rice: 30 weight part, Yulmu: 15 weight part, barley: 20 weight part),

Seeds (wild sesame: 7 weight part, black soybean: 8 weight part, black sesame: 7 weight part),

Dry powders of the compound isolated from the fraction of Example <2-3> (3 weight part),

Ganoderma lucidum (0.5 weight part),

Rehmannia glutinosa (0.5 weight part)

<2-8> Preparation of health foods

| Fraction of Example <2-3> | 1000 | mg |
|---|---|---|
| Vitamin complex | proper amount | |
| Vitamin A acetate | 70 | μg |
| Vitamin E | 1.0 | mg |
| Vitamin B1 | 0.13 | mg |
| Vitamin B2 | 0.15 | mg |
| Vitamin B6 | 0.5 | mg |
| Vitamin B12 | 0.2 | μg |
| Vitamin C | 10 | mg |
| Biotin | 10 | μg |
| Nicotinic acid amide | 1.7 | mg |
| Folic acid | 50 | μg |
| Calcium pantothenate | 0.5 | mg |
| Minerals | proper amount | |
| Ferrous sulfate | 1.75 | mg |
| Zinc oxide | 0.82 | mg |
| Magnesium carbonate | 25.3 | mg |
| Potassium phosphate monobasic | 15 | mg |
| Potassium phosphate dibasic | 55 | mg |
| Potassium citrate | 90 | mg |
| Calcium carbonate | 100 | mg |
| Magnesium chloride | 24.8 | mg |

Vitamins and minerals were mixed according to the preferable composition rate for health food. However, the composition rate can be adjusted. The constituents were mixed according to the conventional method for preparing health food and then the composition for health food was prepared according to the conventional method.

### MANUFACTURING EXAMPLE 3

### Preparation of Beverages

<3-1> Preparation of health beverages

| | | |
|---|---|---|
| Fraction of Example <2-4> | 1000 | mg |
| Citric acid | 1000 | mg |
| Oligosaccharide | 100 | g |
| Maesil (Prunus mume) Extract | 2 | g |
| Taurine | 1 | g |
| Purified water | up to 900 | ml |

The above constituents were mixed according to the conventional method for preparing health beverages. The mixture was heated at 85° C. for 1 hour with stirring and then filtered. The filtrate was loaded in 2 liter sterilized containers, which were sealed and sterilized again, stored in a refrigerator until they would be used for the preparation of a composition for health beverages.

The constituents appropriate for favorite beverages were mixed according to the preferred mixing ratio but the composition ratio can be adjusted according to regional and national preferences, etc.

### <3-2> Preparation of Vegetable Juice

Health enhancing vegetable juice was prepared by adding 5 g of the extract of Example <1-5> of the present invention to 1,000 ml of tomato or carrot juice according to the conventional method.

### <3-3> Preparation of Fruit Juice

Health enhancing vegetable juice was prepared by adding 1 g of the extract of Example <1-5> of the present invention to 1,000 ml of apple or grape juice according to the conventional method.

Those skilled in the art will appreciate that the conceptions and specific embodiments disclosed in the foregoing description may be readily utilized as a basis for modifying or designing other embodiments for carrying out the same purposes of the present invention. Those skilled in the art will also appreciate that such equivalent embodiments do not depart from the spirit and scope of the invention as set forth in the appended claims.

1-9. (canceled)

10. A method for reducing inflammation comprising administering a pharmaceutically effective amount of an extract of Magnolia obovata to a subject having inflammation.

11-18. (canceled)

19. The method of claim 10, wherein the Magnolia obovata is Magnolia obovata Thunberg (Magnoliaceae).

20. The method of claim 10, wherein the extract is extracted from Magnolia obovata fruits or floral buds.

21. The method of claim 10, wherein the extract is extracted by using C₁-C₄ lower alcohol or alcohol aqueous solution as a solvent.

22. The method of claim 21, wherein the lower alcohol is ethanol or methanol.

23. The method of claim 10, wherein the inflammation causes arthritis.

24. The method of claim 10, wherein the inflammation is caused by over-production of nitric oxide.

25. The method of claim 23, wherein macrophage cells produce the nitric oxide.

26. The method of claim 10, wherein the extract of Magnolia obovata contains obovatol represented by Formula 1, honokiol represented by Formula 2 and magnolol represented by Formula 3:

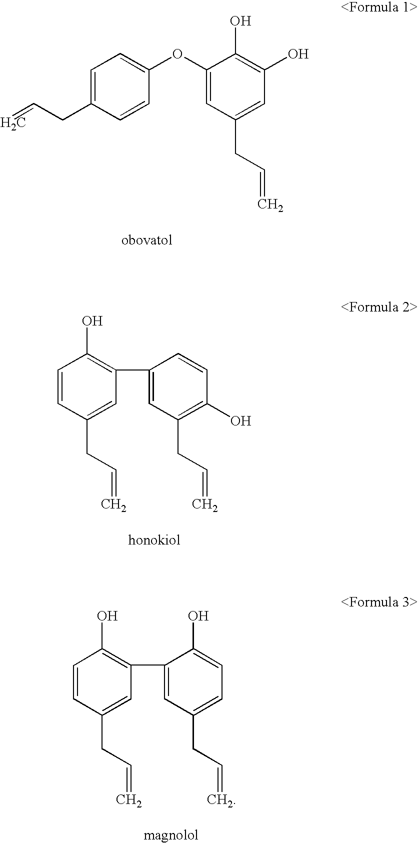

27. The method of claim 10, wherein the subject is a human or non-human mammal.
